(19) United States
(12) Patent Application Publication
(10) Pub. No.: US 2016/0083313 A1
(43) Pub. Date: Mar. 24, 2016

(54) PROCESS FOR CONVERSION OF LIGHT ALIPHATIC HYDROCARBONS TO AROMATICS

(71) Applicant: UOP LLC, Des Plaines, IL (US)

(72) Inventors: Antoine Negiz, Wilmette, IL (US); James E. Rekoske, Glenview, IL (US)

(21) Appl. No.: 14/494,722

(22) Filed: Sep. 24, 2014

(51) Int. Cl. C07C 2/42; C07C 2/00; C07C 2/66

(52) U.S. Cl. CPC C07C 2/42; C07C 2/66; C07C 2/00; C07C 2529/06

(57) ABSTRACT

A process is disclosed for the aromatization and alkylation of light aliphatic hydrocarbons, such as propane or propylene, into aromatic hydrocarbons. The process provides increased aromatics production and decreases methane and ethane production. This improvement for the aromatization and alkylation of light aliphatic hydrocarbons is achieved by adding a benzene stream to the light paraffin feed components.

### FIELD

The present subject matter relates generally to methods and apparatuses for hydrocarbon conversion. More specifically, the present subject matter relates to methods and apparatuses for a catalytic process referred to as dehydrocyclodimerization wherein two or more molecules of a light aliphatic hydrocarbon, such as propane or propylene, are joined together to form a product aromatic hydrocarbon.

### BACKGROUND

Dehydrocyclo-oligomerization is a process in which aliphatic hydrocarbons are reacted over a catalyst to produce aromatics and hydrogen and certain byproducts. This process is distinct from more conventional reforming where C₆ and higher carbon number reactants, primarily paraffins and naphthenes, are converted to aromatics. The aromatics produced by conventional reforming contain the same or a lesser number of carbon atoms per molecule than the reactants from which they were formed, indicating the absence of reactant oligomerization reactions. In contrast, the dehydrocyclo-oligomerization reaction results in an aromatic product that typically contains more carbon atoms per molecule than the reactants, thus indicating that the oligomerization reaction is an important step in the dehydrocyclo-oligomerization process. Typically, the dehydrocyclo-oligomerization reaction is carried out at temperatures in excess of 260° C. using dual functional catalysts containing acidic and dehydrogenation components.

Aromatics, hydrogen, a C₄₊ nonaromatics byproduct, and a light ends byproduct are all products of the dehydrocyclo-oligomerization process. The aromatics are the desired product of the reaction as they can be utilized as gasoline blending components or for the production of petrochemicals. Hydrogen is also a desirable product of the process. The hydrogen can be efficiently utilized in hydrogen consuming refinery processes such as hydrotreating or hydrocracking processes. The least desirable product of the dehydrocyclo-oligomerization process is light ends byproducts. The light ends byproducts consist primarily of C₁ and C₂ hydrocarbons produced as a result of the hydrocracking side reactions.

Traditionally, the dehydrocyclodimerization process includes a combined reactor feed having only the feed and recycled light paraffin feed components.

Accordingly, it is desirable to develop methods for dehydrocyclodimerization with improved yields and selectivity. Furthermore, other desirable features and characteristics of the present embodiment will become apparent from the subsequent detailed description and the appended claims, taken in conjunction with the accompanying drawings and this background.

### SUMMARY

Methods for hydrocarbon conversion are provided. In one approach, a method includes a process flow which increases the yield of more valuable alkylaromatic hydrocarbons in a dehydrocyclodimerization process. It has also been discovered that the invention yields the unexpected result of a higher per pass conversion of the feed of light hydrocarbons. The invention is characterized by the addition of benzene into the dehydrocyclodimerization reaction zone in admixture with feed light hydrocarbons.

A broad embodiment of the invention may be characterized as a process which comprises the steps of passing a first process stream comprising benzene and a feed stream comprising a C₂-C₅ aliphatic feed hydrocarbon into a reaction zone maintained at dehydrocyclodirnerization conditions and containing a solid catalyst and producing a reaction zone effluent stream which comprises the feed hydrocarbon, benzene, toluene and xylenes; and, separating the reaction zone effluent stream in a separation zone and producing a product stream comprising xylenes.

The addition of the benzene feed results in a combined C₁ and C₂ product selectivity of less than 19%, and a selectivity of the aromatic product of more than 50%.

An advantage of the hydrocarbon conversion process is that the aromatics production is increased.

Another advantage of the methods hydrocarbon conversion process is that the production of methane and ethane is decreased.

Another advantage of the hydrocarbon conversion process is that the combined selectivity of C₁ and C₂ hydrocarbons is less than 19%.

A further advantage of the hydrocarbon conversion process is that the selectivity of the aromatic product is more than 50%.

Yet another advantage of the hydrocarbon conversion process is that aromatics which are known precursors of xylenes, such as toluene and heavier aromatics can be produced with high selectivity when benzene is recycled.

Another advantage of the hydrocarbon conversion process is that C₈₊ aromatics which are even more preferred precursors of xylenes can be produced with high selectivity when benzene and toluene are recycled.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### DEFINITIONS

As used herein, the term “dehydrocyclodimerization” is also referred to as aromatization of light paraffins. Within the subject disclosure, dehydrocyclodimerization and aromatization of light hydrocarbons are used interchangeably.

As used herein, the term “stream”, “feed”, “product”, “part” or “portion” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and non-aromatic hydrocarbons. Moreover, the hydrocarbon molecules may be abbreviated C₁, C₂, C₃. Cn where “n” represents the number of carbon atoms in the one or more hydrocarbon molecules or the abbreviation may be used as an adjective for, e.g., non-aromatics or compounds. Similarly, aromatic compounds may be abbreviated A₆, A₇, A₈. An where “n” represents the number of carbon atoms in the one or more aromatic molecules. Furthermore, a superscript “+” or “−” may be used with an abbreviated one or more hydrocarbons notation, e.g., C₃₊ or C₃₋, which is inclusive of the abbreviated one or more hydrocarbons. As an example, the abbreviation “C₃₊” means one or more hydrocarbon molecules of three or more carbon atoms.

As used herein, the term “zone” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

As used herein, the term “aromatic alkylating agent” means a non-aromatic compound or radical used to produce higher alkyl substituted one or more aromatic compounds. Examples of one or more non-aromatic compounds can include an alkane or a cycloalkane, preferably at least one C₂-C₈ alkane or C₅₊ cycloalkane. A non-aromatic radical can mean a saturated group forming a linear or branched alkyl group, a cycloalkyl, or a saturated group fused to an aromatic ring. Aromatic compounds having such non-aromatic radicals can include cumene, indane, and tetralin. The alkylated aromatic compounds can include additional substituent groups, such as methyl, ethyl, propyl, and higher groups. Generally, an aromatic alkylating agent includes atoms of carbon and hydrogen and excludes hetero-atoms such as oxygen, nitrogen, sulfur, phosphorus, fluorine, chlorine, and bromine.

As used herein, the term “rich” can mean an amount of at least generally 50%, and preferably 70%, by mole, of a compound or class of compounds in a stream.

As used herein, the term “substantially” can mean an amount of at least generally 80%, preferably 90%, and optimally 99%, by mole or weight, of a compound or class of compounds in a stream.

As used herein, the term “active metal” can include metals selected from IUPAC Groups that include 7, 8, 9, 10, and 13 such as rhenium, platinum, palladium, rhodium, iridium, ruthenium, osmium, gallium, and indium.

As used herein, the term “modifier metal” can include metals selected from IUPAC Groups that include 11-17. The IUPAC Group 11 trough 17 includes without limitation sulfur, gold, tin, germanium and lead.

### BRIEF DESCRIPTION OF THE DRAWINGS

The drawing FIGURES depict one or more implementations in accord with the present concepts, by way of example only, not by way of limitations. In the FIGURES, like reference numerals refer to the same or similar elements.

FIG. 1 is a schematic depiction of an exemplary aromatic production process and apparatus in accordance with various embodiments for the production of C₈ aromatics from propane and benzene being fed into the reaction zone.

### DETAILED DESCRIPTION

The following detailed description is merely exemplary in nature and is not intended to limit the application and uses of the embodiment described. Furthermore, there is no intention to be bound by any theory presented in the preceding background or the following detailed description.

The various embodiments described herein relate to methods and apparatuses for hydrocarbon conversion. More specifically, the present invention relates to methods and apparatuses for a catalytic process referred to as dehydrocyclodimerization wherein two or more molecules of a light aliphatic hydrocarbon, such as, for example, propane or propylene, are joined together to form a product aromatic hydrocarbon. The basic utility of the process in the conversion of the low cost and highly available light aliphatic hydrocarbons, for example, C₃ and C₄ hydrocarbons, into more valuable aromatic hydrocarbons and hydrogen or to convert the feed hydrocarbons to higher molecular weight aliphatic products. This may be desired simply to upgrade the value of the hydrocarbons. It may also be desired to capitalize on a large supply of the C₃ and C₄ hydrocarbons or to fulfill a need for the aromatic hydrocarbons. The aromatic hydrocarbons produced can be used for various applications, including in the production of a wide range of petrochemicals, including benzene, a widely used basic feed hydrocarbon chemicals. The product aromatic hydrocarbons are also useful as blending components in high octane number motor fuels.

In accordance with one aspect, the feed compounds to a dehydrocyclodimerization process include light aliphatic hydrocarbons having from 2 to 4 carbon atoms per molecule. The feed stream may comprise only one of C₂, C₃, and C₄ compounds or a mixture of two or more of these compounds. In one example, the feed compounds include one or more of propane, propylene, butanes, and the butylenes, with saturates being highly preferred. The feed stream to the process may also contain some C₅ hydrocarbons. In one approach, the concentration of C₅ hydrocarbons in the feed stream to a dehydrocyclodimerization process is held to a minimum practical level, preferably below 5 mole percent. By one aspect, the products of the process include C₆-plus aromatic hydrocarbons. In addition to the desired C₆-plus aromatic hydrocarbons, some nonaromatic C₆-plus hydrocarbons may be produced, even from saturate feeds. When processing a feed made up of propane and/or butanes, the a large portion of the C₆-plus product hydrocarbons will be benzene, toluene, and the various xylene isomers. A small amount of C₉-plus aromatics may also be produced. Where the feed stream includes olefins, an increased production of C₆-plus long chain hydrocarbons may occur as products with the preferred catalyst system. Sizable olefin concentrations in the feed decreases the production of aromatics. By one aspect, the amount of olefins in the feed stream is restricted.

The light aromatic stream may be rich in benzene, methylbenzene (also known as toluene), and mixtures thereof. The aromatic product comprises aromatics that are present after the reaction zone, which are then separated as products in the separation zone. For example if the co-fed light aromatic stream is rich in benzene then the aromatic product will be substantially rich in toluene and heavier aromatics.

The process can be co-fed a light aromatic stream which is substantially rich in methylbenzene or rich in both benzene and methylbenzene. In these instances one skilled in the art will understand that the aromatic product can be adjusted for improved process performance by including suitable amounts of benzene, methyl benzene and heavier aromatics, as individual stream or as mixtures thereof.

In accordance with one aspect, the process includes increasing the amount of the more valuable C₈ alkylaromatics, specifically xylenes, which are produced in a dehydrocyclodimerization reaction zone. By way of example and not limitation, a suitable system for carrying out the processes described herein includes a moving bed radial flow multi-stage reactor such as is described in U.S. Pat. Nos. 3,652,231; 3,692,496; 3,706,536; 3,785,963; 3,825,116; 3,839,196; 3,839,197; 3,854,887; 3,856,662; 3,918,930; 3,981,824; 4,094,814; 4,110,081; and 4,103,909. The systems that may be used in the present process may also include regeneration systems and various aspects of moving catalyst bed operations and equipment as described in these patents. This reactor system has been widely employed commercially for the reforming of naphtha fractions. Its use has also been described for the dehydrogenation of light paraffins.

The reaction zone operates under light aliphatic aromatization and alkylation conditions. Therefore the reaction zone operating conditions promote both the formation of aromatics from light hydrocarbons such as C2-C8 paraffins, naphthenes, as well as the alkylation of activated intermediate forms of these molecules onto the aromatic components present including benzene, methylbenzene, and other heavier aromatics as well.

Conditions for aromatization of light hydrocarbons are known to favor low pressures and high temperatures. Hence for the dehydrocyclodimerization typical conditions are described in U.S. Pat. No. 4,642,402 A. The preferred metallic component is gallium as described in the previously cited U.S. Pat. No. 4,180,689. The balance of the catalyst can be composed of a refractory binder or matrix that is optionally utilized to facilitate fabrication, provide strength, and reduce costs. Suitable binders can include inorganic oxides, such as at least one of alumina, magnesia, zirconia, chromia, titania, boria, thoria, phosphate, zinc oxide and silica.

Aromatization and alkylation conditions, according to the present invention, include temperatures 450 C to 600 C. In another approach, the aromatization and alkylation conditions may include a temperature between about 950 degree F. and 1050 degree F. (487 degree and 565 degree C.).

Aromatization and alkylation conditions, according to the present example, include pressures between 17 Psig to 200 Psig. In one approach, the aromatization and alkylation conditions may include pressures under 100 psig (689 kPag). The aromatization and alkylation conditions in another approach include a pressure between 50 Psig and 150 Psig. Without being limited by theory, hydrogen-producing reactions are normally favored by lower pressures, and accordingly in one approach conditions may include a pressure under about 70 psig (483 kPag) at the outlet of the reaction zone.

The aromatization and alkylation conditions may include hydrogen at the reactor inlet. For example, hydrogen may be present in the feed streams to the reaction zone or separately introduced. Typically the addition of hydrogen may improve catalyst stability. It should be noted that hydrogen is not essential to the processes disclosed herein. In one example, hydrogen may be present in an amount equivalent to a hydrogen to hydrocarbon mole ratio of a total feed from 0.01 to 0.5, and in another example from 0.05 to 0.3.

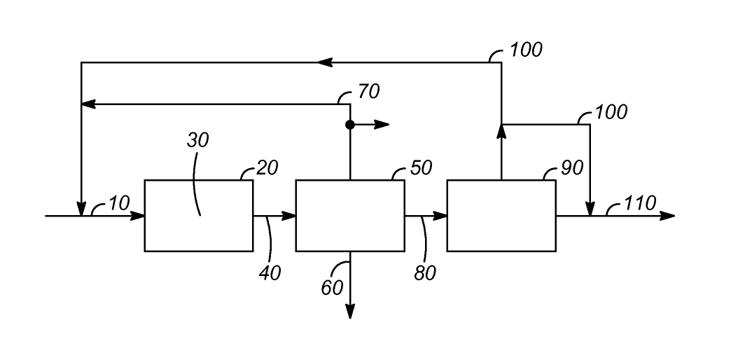

FIG. 1 illustrates a flow diagram of various embodiments of the processes described herein. Those skilled in the art will recognize that this process flow diagram has been simplified by the elimination of many pieces of process equipment including for example, heat exchangers, process control systems, pumps, fractionation column overhead and reboiler systems, etc. which are not necessary to an understanding of the process. It may also be readily discerned that the process flow presented in the drawing may be modified in many aspects without departing from the basic overall concept. For example, the depiction of required heat exchangers in the drawing have been held to a minimum for purposes of simplicity. Those skilled in the art will recognize that the choice of heat exchange methods employed to obtain the necessary heating and cooling at various points within the process is subject to a large amount of variation as to how it is performed. In a process as complex as this, there exists many possibilities for indirect heat exchange between different process streams. Depending on the specific location and circumstance of the installation of the subject process, it may also be desired to employ heat exchange against steam, hot oil, or process streams from other processing units not shown on the drawing.

With reference to FIG. 1, a system and process in accordance with various embodiments includes a reaction zone 20. A feed stream 10 enters the reaction zone 20. The reaction zone 20 operates under typical aromatization of light hydrocarbon and alkylation conditions in the presence of a typical aromatization of light hydrocarbon and alkylation catalyst 30 and produces a reaction zone product stream 40. The reaction zone 20 can include one or more reactor vessels that contain an aromatization and alkylation catalyst. These reactors can further be connected with and without additional separation equipment, and they may be connected in series or in parallel. The reaction zone 20 may generate at least one outlet stream 40. The reaction zone outlet stream 40 may be sent to a separation zone.

As discussed previously, the feed stream 10 includes light aliphatic compounds and aromatic compounds. The feed stream 10 may be introduced as a combined feed stream or may include two or more separate stream, for example a light aliphatic compound stream and an aromatic compound stream, that are introduced to the reaction zone 20 separately. Light aliphatic compound streams can be introduced to the reaction zone 20 in a form that could be liquid or vapor or a mixture thereof. By way of one example, the fresh portion of a C₃-C₄ aliphatic feed may be available in liquid form (liquefied petroleum gas). On the other hand the light aliphatic recycle stream 70 as recovered from zone 50 could be in a form that is vapor or liquid or a mixture thereof.

Any suitable catalyst may be utilized such as at least one molecular sieve including any suitable material, e.g., alumino-silicate. The catalyst can include an effective amount of the molecular sieve, which can be a zeolite with at least one pore having a 10 or higher member ring structure and can have one or higher dimension. Typically, the zeolite can have a Si/Al₂ mole ratio of greater than 10:1, preferably 20:1-60:1. Preferred molecular sieves can include BEA, MTW, FAU (including zeolite Y in both cubic and hexagonal forms, and zeolite X), MOR, LTL, ITH, ITW, MEL, FER, TON, MFS, IWW, MFI, EUO, MTT, HEU, CHA, ERI, MWW, AEL, AFO, ATO, and LTA. Preferably, the zeolite can be MFI and/or MTW. Suitable zeolite amounts in the catalyst may range from 1-100%, and preferably from 10-90%, by weight.

Generally, the catalyst includes at least one metal selected from active metals, and optionally at least one metal selected from modifier metals. The total active metal content on the catalyst by weight is about less than 2% by weight. In some embodiments, the preferred total active metal content is less than about 1.5%, in yet in another embodiments the preferred total active metal content is less than 1%, still in yet in another embodiment the total active metal content on the catalyst by weight is less than 0.5 wt %. At least one metal is selected from IUPAC Groups that include 7, 8, 9, 10, and 13. The IUPAC Group 7 trough 10 includes without limitation rhenium, platinum, palladium, rhodium, iridium, ruthenium and osmium. The IUPAC Group 13 includes without limitation gallium, indium. In addition to at least one active metal, the catalyst may also contain at least one modifier metal selected from IUPAC Groups 11-17. The IUPAC Group 11 trough 17 includes without limitation sulfur, gold, tin, germanium and lead.

In the example illustrated in FIG. 1, the reaction zone product stream 40 is sent to a light product separation zone 50 where one or more streams are generated. In this example, the light product separation zone 50 produces a first outlet stream 60, a second outlet stream 70, and a third outlet stream 80. The first light product separation zone outlet stream 60 contains hydrogen, C₁, and C₂ hydrocarbons. The second light product separation zone outlet stream 70 is rich in C₂-C₅ hydrocarbons, which may include a purge of the C₂-C₅ hydrocarbons, but also recycles the C₂-C₅ hydrocarbons to be mixed with the feed 10. The third light product separation zone outlet stream 80 contains C₆+ aromatics and is sent to the aromatic product separation zone 90. The light product separation zone 50 may have multiple separation vessels, each having multiple outlet streams comprising hydrogen, C₁-C₂ hydrocarbons, and C₂-C₅ hydrocarbons. These vessels may include but not limited to flash drums, condensers, reboilers, trayed or packed towers, distillation towers, adsorbers, cryogenic loops, compressors, and combinations thereof.

Turning back to FIG. 1, stream 80 contains C₆+ aromatics and is sent to the aromatic product separation zone 90. The aromatic product separation zone 90 generates more product streams. In this embodiment, the aromatic product separation zone 90 produces an outlet stream 100 and a product stream 120. The outlet stream 100 includes C₆-C₇ hydrocarbons. The outlet stream 100 may be further separated, it may be recycled, or it may be sent to the product stream 110. In the example shown in FIG. 1, a portion of the outlet stream 100 is recycled back to the reaction zone 20 such that the C₆-C₇ hydrocarbons are mixed with the feed 10 and a portion of the outlet stream 100 is mixed with the product stream 110. The product stream 110 is rich in C₈₊ aromatics, but also contains some C₆-C₇ aromatics from the outlet stream 100. It is contemplated that the aromatic product separation zone 90 may have multiple separation vessels, each having multiple outlet streams comprising C₆-C₇ hydrocarbons, and C₈₊ aromatics. These vessels may include but not limited to flash drums, condensers, reboilers, trayed or packed towers, distillation towers, adsorbers, cryogenic loops, compressors, and combinations thereof.

The addition of the benzene to the feed results in a combined selectivity of C₁ and C₂ hydrocarbons that is less than 19%, and a selectivity of the aromatic product of more than 50%.

### Examples

The following examples are intended to further illustrate the subject embodiments. These illustrations of embodiments of the invention are not meant to limit the claims of this invention to the particular details of these examples. These examples are based on pilot plant data.

Both runs are simulated at generally the same conditions, such as at a pressure of about 50 psig, a temperature of 500° C., and a weight hourly space velocity of 2. The composition in percent, by weight, of the feed and product runs as well as the results are depicted in Table 1 below. An example catalyst was prepared and used to demonstrate the impact of the claimed subject matter. 10 grams of fresh catalyst was loaded for each of the two tests with the two different feeds in the tables labeled below as Feed 1 and 2.

For each run, 10 grams catalyst was loaded in a standard fixed bed reactor with a thermowell, capable of measuring temperatures in fixed locations inside the catalyst bed. In what follows, the average is referred to as the average bed temperature or reactor temperature. The hydrocarbon (HC) feed compositions by weight for each test are as listed in Table 1, as feed 1 for the first test, and feed 2 for the second test.

For each test the catalyst was pretreated under first nitrogen flow for drying the catalyst. This followed by a hydrogen pretreatment to activate the active metals. After the hydrogen pretreatment the flow was switched back to nitrogen and the catalyst bed was cooled down to the first reactor temperature test setting, 500 C. Hydrocarbon liquid feed at the specified compositions was then introduced, and the nitrogen flow was stopped. Both tests were conducted at the same conditions at a pressure of about 50 psig, and a weight hourly space velocity of 2. At the test conditions at the inlet of the reactor the fluid is expected to be substantially rich in vapor. During each test, after the 500 C first reactor temperature condition testing was completed, several additional test periods at higher temperature conditions and also with some other feed compositions were also conducted. Table 1 and 2 shows the 500 C reactor temperature results.

The reactor effluent composition was generated by combining the compositions and mass flows measured of two product streams recovered as gas and liquid downstream of the reactor. The total reactor effluent hydrocarbon composition was then obtained by normalizing the merged hydrocarbon component mass flows to 100%. The liquid product and gas product compositions were obtained using two on-line GC systems, analyzing the gas and the liquid approximately once every hour.

TABLE 1

| | FEED 1 | PRODUCT 1 | FEED 2 | PRODUCT 2 |
|---|---|---|---|---|
| C1 | 0.0 | 11.5 | 0.0 | 4.2 |
| C2 | 0.0 | 12.6 | 0.0 | 3.6 |
| C3 | 71.9 | 35.7 | 36.0 | 12.1 |
| n-C4 | 27.7 | 1.4 | 13.8 | 0.5 |
| i-C4 | 0.1 | 0.6 | 0.0 | 0.0 |
| n-C5 | 0.0 | 0.1 | 0.0 | 0.0 |
| i-C5 | 0.3 | 0.1 | 0.2 | 0.0 |
| C6-C8 non-A | 0.0 | 0.1 | 0.0 | 0.0 |
| BZ | 0.0 | 6.3 | 50.0 | 35.3 |
| TOL | 0.0 | 14.1 | 0.0 | 21.5 |
| EB | 0.0 | 0.4 | 0.0 | 1.6 |
| XY | 0.0 | 9.4 | 0.0 | 6.3 |
| C9A Plus | 0.0 | 7.6 | 0.0 | 14.8 |
| TOTAL | 100 | 100 | 100 | 100 |
| C1 + C2 | | 24.1 | | 7.9 |
| A7Plus | | 31.6 | | 44.1 |

As depicted in Table 1, feed 1 did not include benzene, whereas feed 2 included a benzene feed of 50. The products resulting from feed 1 resulted in a weight percent of C₁-C₂ at 24, whereas feed 2 did include a benzene feed and produced only 8 weight percent of C₁-C₂. Further, the weight percent of A₇₊ aromatics went from 32 weight percent to 44 weight percent with the added benzene feed.

Furthermore, selectivity may be used to calculate and further illustrate the performance data provided in Table 1. The selectivity calculation results are depicted in Table 2 below:

TABLE 2

| | PRODUCT 1 | | PRODUCT 2 |
|---|---|---|---|
| % Conversions | | % Conversions | |
| Overall | 62.7 | Overall | 52.1 |
| C4&C3 | 62.2 | C4&C3 | 74.7 |
| Benzene | NA | Benzene | 29.4 |
| % Aromatic | NA | % Aromatic | 136.1 |
| Recovery | | Recovery | |
| % Selectivities | | % Selectivities | |
| C1 | 18.4 | C1 | 8.1 |
| C2 | 20.1 | C2 | 7.0 |
| C3 | 0.0 | C3 | 0.0 |
| n-C4 | 0.0 | n-C4 | 0.0 |
| i-C4 | 0.8 | i-C4 | 0.0 |
| n-C5 | 0.1 | n-C5 | 0.0 |
| i-C5 | 0.0 | i-C5 | 0.0 |
| C6-C8 non-A | 0.1 | C6-C8 non-A | 0.1 |
| BZ | 10.1 | BZ | 0.0 |
| TOL | 22.5 | TOL | 41.2 |
| EB | 0.6 | EB | 3.0 |
| XY | 15.0 | XY | 12.2 |
| C9A Plus | 12.2 | C9A Plus | 28.4 |
| TOTAL | 100 | TOTAL | 100 |
| C1 + C2 | 38.5 | C1 + C2 | 15.1 |
| A7Plus | 50.3 | A7Plus | 84.8 |

As depicted in Table 2, feed 1 did not include benzene, whereas feed 2 included a benzene feed. The products resulting from feed 1 resulted in a C₁-C₂ selectivity of 38.504, whereas feed 2 did include a benzene feed and resulted in a C₁-C₂ selectivity of 15.122. Further, the selectivity of A₇₊ aromatics went from 50.329 to 84.768 with the added benzene feed. Furthermore the aromatic mole ring recovery of the feed 2 product with respect to feed 2 is 136%, which is greater than 100%. Without bound to any theory, one skilled in the art referring to the data presented in Tables 1 and 2 can deduce the presence of light aliphatic aromatization and alkylation especially in the example case for feed 2.

It should be noted that various changes and modifications to the presently preferred embodiments described herein will be apparent to those skilled in the art. Such changes and modifications may be made without departing from the spirit and scope of the present invention and without diminishing its attendant advantages.

1. A method of hydrocarbon conversion comprising:
feeding a vapor phase feed stream comprising a light aliphatic hydrocarbon stream and a light aromatic stream to a reaction zone comprising a catalyst and contacting the feed stream with the catalyst to form a reaction zone effluent stream comprising an aromatic product.

2. The method of claim 1 wherein the light aliphatic hydrocarbon stream is rich in at least one of C₃ hydrocarbons, C₄ hydrocarbons, or a combination thereof.

3. The method of claim 1 wherein the light aliphatic hydrocarbon stream is rich in C₄ hydrocarbons.

4. The method of claim 1 wherein feeding the vapor phase feed stream comprising light aliphatic hydrocarbons to the reaction zone comprises feeding at least a portion of the reaction zone effluent stream comprising light aliphatic hydrocarbons, at least a portion of the reaction zone effluent stream comprising light aromatic hydrocarbons, and a fresh feed stream comprising light aliphatic and light aromatic hydrocarbons to the reaction zone.

5. The method of claim 1 wherein the reaction zone comprises at least one reactor.

6. The method of claim 1, wherein the pressure of the reaction zone is between about 17 to about 200 Psig.

7. The method of claim 1 wherein the catalyst comprises a zeolite.

8. The method of claim 1 wherein the catalyst comprises at least one active metal.

9. The method of claim 8 wherein the catalyst comprises less than 1.5% gallium.

10. The method of claim 1, wherein the reaction zone effluent stream comprises benzene, toluene, xylenes, and heavier aromatics.

11. The method of claim 1, wherein the concentration of benzene in the hydrocarbons entering the reactor is about 5 to about 95 weight percent.

12. The method of claim 1, wherein the combined selectivity of C₁ and C₂ hydrocarbons is less than 19%.

13. The method of claim 1, wherein the selectivity of the aromatic product is more than 50%.

14. A light aliphatic aromatization and alkylation method comprising:
feeding a vapor phase feed stream comprising at least one of propane and butane and a benzene stream, wherein the benzene stream comprises about 5 to about 95 weight percent of the total feed to a reactor comprising a zoelite catalyst comprising between about 0.25 weight % and 1.5 weight % gallium to form a reactor effluent stream comprising benzene, toluene, xylenes, and heavier aromatics.

15. A dehydrocyclodimerization method comprising:
reacting a vapor phase feed stream comprising a light aliphatic hydrocarbon and a light aromatic stream at light aliphatic aromatization and alkylation conditions to form an effluent stream comprising benzene, toluene, xylenes, and heavier aromatics.

16. The method of claim 15 further comprising reacting the vapor phase feed stream and the light aromatic stream in the presence of a catalyst comprising a zeolite and at least one active metal.

17. The method of claim 16 wherein the catalyst comprises between about 0.25 weight % and 1.5 weight % gallium.

18. The method of claim 15, wherein the concentration of benzene in the light aromatic hydrocarbons entering the reactor is about 5 to about 100 weight percent.

19. The method of claim 15, wherein the combined selectivity of C₁ and C₂ hydrocarbons is less than 19%.

20. The method of claim 15, wherein the selectivity of the aromatic product is more than 50%.
